(19) United States
(12) Patent Application Publication
(10) Pub. No.: US 2019/0358192 A1
(43) Pub. Date: Nov. 28, 2019

(54) SULFUR-BASED BROAD SPECTRUM ANTIVIRALS

(71) Applicant: Washington State University, Pullman, WA (US)

(72) Inventors: Hector Aguilar-Carreno, Ithaca, NY (US); Ming Xian, Pullman, WA (US); Armando Pacheco, Ithaca, NY (US)

(21) Appl. No.: 16/534,388

(22) Filed: Aug. 7, 2019

Related U.S. Application Data
(63) Continuation of application No. 15/928,633, filed on Mar. 22, 2018.
(60) Provisional application No. 62/475,439, filed on Mar. 23, 2017.

(51) Int. Cl. A61K 31/265; A61P 31/12

(52) U.S. Cl. CPC A61K 31/265; A61P 31/12

(57) ABSTRACT

Methods of using the formulations disclosed herein to treat or prevent a broad spectrum of enveloped viruses are provided. In particular, a method of treating or preventing a disease state caused by an enveloped type virus can include agents of a formula wherein R₁, is selected from the group consisting of OH, OR₆, NHR₆, and NR₆R₇ where R₆ and R₇ are selected from the group consisting of alkyl, aryl, methyl and heteroaryl; R₂ is selected from the groups consisting of OR₆, acyl, alky, aryl, and sulfonyl; R₃ is selected from the group consisting of OR₆ alkyl, aryl, substituted aryl, and heteroaryl; R₄ and R₅ are independently selected from the groups consisting of hydrogen, methyl or alkyl, substituted alkyl, aryl, and substituted aryl; and whereby, administration of the compound allows for the treatment of enveloped viruses.

### CROSS-CROSS-REFERENCE TO RELATED APPLICATION(S)

The present application claims under 35 U.S.C. § 119, the priority benefit of U.S. Provisional Application No. 62/475,439, entitled, “SULFUR-BASED BROAD SPECTRUM ANTIVIRALS,” filed Mar. 23, 2017. The disclosure of the foregoing application is incorporated herein by reference in its entirety.

### GOVERNMENT INTERESTS

This invention was made with government support under Grant/Contract Numbers R01HL116571 and R01AI109022, awarded by the National Institutes of Health. The government has certain rights in the invention.

### FIELD OF THE INVENTION

The present embodiments provide various formulations, which act as broad-spectrum antivirals for the treatment and/or the prevention of viral pathogens. In particular, the embodiments herein relate to methods for the treatment or prevention of enveloped typed viruses.

### BACKGROUND OF THE INVENTION

### Discussion of the Related Art

Antiviral medications are traditionally designed to block specific stages of the viral life cycle by viral receptor binding, penetration, nucleic acid chain elongation, transcription, translation, assembly, and release. While all of such traditional implementations of antiviral medications have uses in combating viral infection resulting from pathogens, they also encounter drawbacks and limitations for specific viral types.

The 2015 World Health Organization (WHO) meeting in Geneva produced a list of the eight top emerging pathogens most likely to cause major epidemics world-wide, requiring urgent research and development. Noticeably, all eight pathogens are enveloped viruses (in alphabetical order: Crimean Congo hemorrhagic fever, Ebola, Marburg, Lassa fever, middle east respiratory syndrome (MERS), severe acute respiratory syndrome (SARS), Nipah, and Rift Valley fever viruses). Other important enveloped viruses that cause important levels of disease in humans and animals are influenza virus, herpes viruses, and human immunodeficiency virus.

The vast majority of such human infectious diseases originate in animals (zoonotic). Additionally, the National Academy of Sciences recently reported that zoonotic diseases are a leading cause for illness and death worldwide, and negatively affect the global economy. The list of zoonotic pathogens reported largely overlaps the WHO list.

The present number of promising broad-spectrum antivirals is small, such as, but not limited to, RIBAVIRIN (ie. guansine nucleosides), LJ001, and ARBIDOL (ie. ethyl-6-bromo-4-[(dimethylamino)methyl]-5-hydroxy-1-methyl-2-[(phenylthio)methyl]-indole-3-carboxylate hydrochloride monohydrate). However, the use of current antivirals presents practical use issues such as secondary effects, the need of light for activation, etc., making them less than ideal for use as broad-spectrum antiviral therapeutic agents, particularly for enveloped viruses.

To illustrate such use issues, LJ001 in particular, needs the presence of light in order to produce singlet oxygen, which in turn inhibits the viral membrane. Moreover, while ARBIDOL has been used against influenza virus in Russia and China, its use in the US is not FDA approved since side effects are unclear in spite of low toxicity claims. In addition, although RIBAVIRIN treatment of hepatitis C and respiratory syncytial virus (RSV) infections has been approved in many countries including the US, data for the clinical efficacy of ribavirin against influenza virus are limited, particularly due to small sample sizes, incomplete trial information, and/or incompatible protocols for meta-analysis. Despite the numerous studies for the use of ribavirin against paramyxoviruses, the mechanism of action is still being elucidated and its broad spectrum antiviral effects have yet to be defined.

Therefore, there is a need in the art for a new class of broad-spectrum antiviral therapeutic drugs capable of treating a plurality of viral infections, given the many infectious viral agents that lack effective vaccine or therapeutic approaches and that have the potential to cause global pandemics. In particular, there is a need for antiviral therapeutic drugs that target a specific step common to broad families of enveloped viruses. The present application is directed to such needs.

### SUMMARY OF THE EMBODIMENTS

The present disclosure may be embodied in a method for the treatment of enveloped viruses. Example embodiments herein are directed to a method of treating or preventing a disease or condition caused by infection with an enveloped virus that includes administering to a subject a therapeutically effective amount of a compound or a pharmaceutically acceptable salt thereof.

One example embodiment of the disclosed invention is directed to a method of treating or preventing a disease state caused by an enveloped type virus comprising administering to a subject a therapeutically effective dose of compound having the chemical formula:

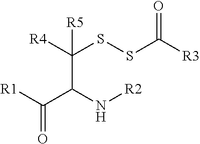

or a pharmaceutically acceptable salt thereof, wherein,

R₁ is selected from the group consisting of OH, OR₆, NHR₆, and NR₆R₇ where R₆ and R₇ are selected from the group consisting of alkyl, aryl, methyl and heteroaryl;

R₂ is selected from the groups consisting of OR₆, acyl, alkyl, aryl, and sulfonyl;

R₃ is selected from the group consisting of OR₆, alkyl, aryl, substituted aryl, and heteroaryl; and

R₄ and R₅ are independently selected from the groups consisting of hydrogen, methyl or alkyl, substituted alkyl, aryl, and substituted aryl.

Another example embodiment of the disclosed invention is directed to a method wherein the compound is administered to the subject in the presence of a pharmaceutically acceptable carrier.

Another example embodiment of the disclosed invention is directed to a method further comprising providing the subject with at least one other therapeutic agent as a combined preparation for simultaneous, separate, or sequential use in therapy.

Another example embodiment of the disclosed invention is directed to a method wherein the second therapeutic agent is in the form of a pharmaceutically acceptable salt.

In another example embodiment, the broad-spectrum antiviral efficacy afforded by the composition and method includes antiviral efficacy against two or more of HIV-1, HIV-2, HTLV-1, and HTLV-2.

In another example embodiment broad-spectrum antiviral efficacy provided by the composition and method may be antiviral efficacy against two or more viruses selected from hepatitis C virus, yellow fever virus, respiratory syncytial virus, Sindbis virus, influenza virus A, Venezuela encephalitis virus, West Nile virus, and Ebola virus.

In another example embodiment, broad-spectrum antiviral efficacy provided by the composition and method may be antiviral efficacy against two or more viruses of hepatitis C virus, yellow fever virus, respiratory syncytial virus, Sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, Ebola virus, Marburg virus, La Crosse virus, California encephalitis virus, Hantaan virus, Crimean-Congo virus, Rift Valley fever, Lassa fever, Argentine hemorrhagic fever virus, Bolivian hemorrhagic fever virus, Colorado tick fever, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

In another example embodiment, the invention provides for an anti-viral agent that is to be used in combination with a therapeutic composition described herein. Anti-viral agents include, but are not limited to abacavir; acemannan; acyclovir; acyclovir sodium; adefovir; alovudine; alvircept sudotox; amantadine hydrochloride; amprenavir; aranotin; arildone; atevirdine mesylate; avridine; cidofovir; cipamfylline; cytarabine hydrochloride; delavirdine mesylate; desciclovir; didanosine; disoxaril; edoxudine; efavirenz; enviradene; enviroxime; famciclovir; famotine hydrochloride; fiacitabine; fialuridine; fosarilate; trisodium phosphonoformate; fosfonet sodium; ganciclovir; ganciclovir sodium; idoxuridine; indinavir; kethoxal; lamivudine; lobucavir; memotine hydrochloride; methisazone; nelfinavir; nevirapine; palivizumab; penciclovir; pirodavir; ribavirin; rimantadine hydrochloride; ritonavir; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; zinviroxime, interferon, cyclovir, alpha-interferon, and/or beta globulin. In certain aspects, antibodies against viral proteins or cellular factors may also be used in combination with a therapeutic composition described herein.

Additionally, a broad-spectrum antiviral drug is administered to a patient as a therapy against an unknown virus or to a patient suspected of having a virus, even though the existence of or type of virus causing the viral infection was unknown. Such an administration of the broad-spectrum antiviral compound at early stages (e.g. a patient suspected of having a virus to include even as a prophylactic treatment) provides for antiviral treatment, which may inhibit replication or an onset and replication of any virus within the patient.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein.

### BRIEF DESCRIPTION OF THE DRAWINGS

To gain an improved understanding of the features of the invention, reference may be made to the following descriptive matter and accompanying figures that describe and illustrate various configurations and concepts related to the invention.

FIG. 1 shows the structures of representative sulfur-containing compounds tested (XM-numbers 01-15). Also shown are the structures of the control compounds LJ001 which is known to inhibit enveloped viruses and GYY4137, which is known to inhibit some paramyxoviruses and pneumoviruses.

FIG. 2A shows compound XM-01 inhibiting pseudotyped NiV infections at low cytotoxicity levels. FIG. 2A in particular shows % infectivity of Vero cells by pseudotyped Nipah virus, wherein compounds were used at 10 μM concentrations.

FIG. 2B shows a second plot of compound XM-01 demonstrating low cytotoxicity levels. The plot in particular shows cell viability following incubation of Vero cells with best compounds from FIG. 2A. Cytotoxicity was determined using a CCK-8 kit to measure dehydrogenase activity of live cells.

FIG. 2C shows a third plot of compound XM-01 inhibiting pseudotyped NiV infections at low cytotoxicity levels. The plot in particular shows XM-01 cytotoxicity at various concentrations between 1 μM-1 mM. (N=3).

FIG. 2D shows a fourth plot of compound XM-01 inhibiting pseudotyped NiV infections at low cytotoxicity levels. The plot in particular shows XM-01 cells in at 0.1% DMSO, along with cells in 2 mM of H2O2 as a negative control.

FIG. 3A shows XM-01 inhibiting, pseudotyped NiV infection of Vero cells at various concentrations. Virions were treated with XM-01 for 30 min before infection. LJ001 is utilized as an inhibitor control while XM-01 is shown inhibiting pseudotyped NiV infections.

FIG. 3B shows XM-01 inhibiting HSV-1. Virions were treated with XM-01 for 30 min before infection. LJ001 is utilized as an inhibitor control while XM-01 is shown inhibiting HSV-1 infections.

FIG. 3C shows XM-01 inhibiting respiratory syncytial virus (RSV), pseudotyped NiV infection of Vero cells at various concentrations. Virions where treated with XM-01 for 30 min before infection. LJ001 is utilized as an inhibitor control while XM-01 is shown inhibiting RSV.

FIG. 3D shows XM-01 inhibiting VSV pseudotyped NiV infection of Vero cells at various concentrations. Virions where treated with XM-01 for 30 min before infection. LJ001 is utilized as an inhibitor control while XM-01 is shown inhibiting VSV.

FIG. 3E shows XM-01 inhibiting Influenza virus. Virions were treated with XM-01 for 30 min before infection. LJ001 is utilized as an inhibitor control while XM-01 is shown inhibiting Influenza virus infections. XM-01 was also shown inhibiting non-enveloped norovirus infections. Virions were treated with XM-01 for 30 min before infection.

FIG. 3F shows XM-01 not inhibiting non-enveloped rotavirus infections. Virions where treated with XM-01 for 30 min before infection. The rotavirus inhibitor control GRA is shown.

FIG. 3G shows XM-01 not inhibiting non-enveloped norovirus infections. Virions where treated with XM-01 for 30 min before infection. The rotavirus inhibitor control GRA is shown.

FIG. 4A shows XM-01 inhibiting virions directly, dilutions of pseudotyped NiV pre-treated with XM-01 for 30 min and washed of excess XM-01 by ultracentrifugation before Vero cell infections. LJ001 was used as an inhibitor control.

FIG. 4B shows Vero cells that were pre-treated with XM-01 for 30 min and washed with warm PBS 3 times, followed by pseudotyped NiV infection, as opposed to treatment of virions with XM-01 and then adding this mixture to cells, and demonstrating the inhibition of virons, and little to no effects on the roles of cells in infections.

FIG. 4C shows the addition of XM-01 at various times post-initial virus exposure to demonstrate the prophylactic and inhibitory nature XM-01 early in the infection process.

FIG. 4D shows the Infection of Vero cells by pseudotyped NiV pre-treated with XM-01 before infecting Vero cells in the darkness. To demonstate that XM-01 does not need light to inhibit the infections, while LJ001 does.

FIG. 5A shows soluble receptor EphrinB2 incubated with cells expressing NiV G glycoprotein for 30 min and shows that XM-01 does not affect receptor binding to G on PK13 cells that do not express the ephrinB2/B3 receptors.

FIG. 5B shows the same conclusion as FIG. 5A but in HEK293T cells that express the ephrinB2/B3 receptors. XM-01 at 10 μM was incubated with cells expressing expressing G glycoprotein and incubated with soluble receptor and XM-01 for 30 min.

FIG. 5C shows that 10 μM of XM-01 does not interfere with the triggering of the NiV F protein in the fusion cascade process.

FIG. 5D shows XM-01 at 10 μM or the control membrane fusion inhibitor LJ001 at 1 μM both inhibit cell to cell fusion.

FIG. 5E shows that neither XM-01 at 10 μM nor LJ001 at 1 μM significantly affect protein G or F expression on cell surfaces.

FIG. 5F shows that neither XM-01 at 10 μNI nor LJ001 at 1 μM significantly affect conformation changes of protein G or F in selected cell lines.

FIG. 6A shows virons treated with DMSO (0.1%) vehicle control, in the electron microscopy images, black arrows indicate affected membranes; white arrows indicate RNA spill. This control experiment shows no significant effects on virus morphology, membranes, or RNA spill.

FIG. 6B shows virons treated with LJ001 membrane inhibitor control, in the electron microscopy images, black arrows indicate affected membranes; white arrows indicate RNA.

FIG. 6C shows virons treated with XM-01, in the electron microscopy images, black arrows indicate affected membranes; white arrows indicate RNA.

FIG. 6D shows Pseudotyped NiV virions treated with XM-01 were then added a fluorescent pyrene lipid probe to monitor their emission of monomers and formation of excimers. The DMSO control virions show a relative constant monomer emission and formation and emission of excimers.

FIG. 6E shows viruses treated with LJ001 the ratio of excimer emission to monomer emission is decreased.

FIG. 6F shows XM-01-treated virions, a severe decrease in the ratio of the emission of excimers to monomers is an indication of lower membrane fluidity.

FIG. 6G shows a comparison of the ratios of excimer to monomer emissions gives us insight on the membrane fluidity levels. Virons treated with DMSO show an increase in the ratio while those treated with XM-01 of LJ001 do not.

### DETAILED DESCRIPTION

The following are definitions of terms that may be used in the present specification. The initial definition provided for a group or term herein applies to that group or term throughout the present specification individually or as part of another group, unless otherwise indicated. Additionally, it will be understood that any list of such candidates or alternatives is merely illustrative, not limiting, unless implicitly or explicitly understood or stated otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “pharmaceutically acceptable excipient” includes buffering agents, preservatives, solvents, diluents, carriers, liquid vehicles, dispersion or suspension aids, surface active agents, isotonic agents, thickening or emulsifying agents, preservatives, solid binders, lubricants and the like which are compatible with the antiviral compounds provided herein and suitable for the particular dosage form desired.

The term “pharmaceutically acceptable salt” means a salt from of a compound with an alkali metal such as sodium, potassium and lithium, with alkaline earth metals such as calcium and magnesium, with organic bases such as dicyclohexylamine, tributylamine, pyridine and amino acids such as arginine, lysine and the like. Compounds may also form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art.

The term “acyl” refers to or denotes a chemical group containing the monovalent group of atoms RCO—, where R is an organic group. Acyl groups contain at least one carboxylic-acid-derived chemical group, e.g. a chemical group derived from a carboxylic acid by removal of a hydroxyl group. In organic chemistry, the acyl group is usually derived from a carboxylic acid (IUPAC name: alkanoyl). Therefore, it has the formula RCO—, where R represents e.g. an alkyl group that is attached to the CO group with a single bond. Representative acyl groups include but are not limited to formyl, acetyl, propionyl, acrylyl, etc. Although the term is almost always applied to organic compounds, acyl groups can in principle be derived from other types of acids such as sulfonic acids, phosphonic acids. In the most common arrangement, acyl groups are attached to a larger molecular fragment, in which case the carbon and oxygen atoms are linked by a double bond.

The term “alkyl” refers to straight or branched chain unsubstituted hydrocarbon groups of 1 to 20 carbon atoms, typical alkyl groups include methyl, ethyl, propyl, isopropyl, butyl, isobutyl, tertiary butyl, pentyl, hexyl and the like

As used herein the term “substituted” refers to a hydrocarbon compound (e.g. an alkyl, alkenyl, alkynyl, ring, etc. structure as described herein) in which an H bonded to a C is replaced or substituted by a different atom or groups of atoms e.g. a saturated 6-membered straight chain in which the 2 terminal C atoms are bonded to three H atoms, three of the four internal C atoms are bonded to 2H atoms, and one of the internal C atoms is bonded to H and also to a different atom or group of atoms (e.g. OH). In this case, the hydrocarbon chain is “substituted” by (with) OH.

The term “aryl” refers to monocyclic or bicyclic aromatic hydrocarbon groups having 6 to 12 carbon atoms in the ring portion, such as phenyl, naphthyl, biphenyl and diphenyl groups, each of which may be substituted.

The term “substituted aryl” refers to an aryl group substituted by, for example, one to four substituents such as alkyl, substituted alkyl, alkenyl, substituted alkenyl, alkynyl, substituted alkynyl, aryl, substituted aryl, aralkyl, halo, trifluoromethoxy, trifluoromethyl, hydroxy, alkoxy, alkanoyl, alkanoyloxy, aryloxy, aralkyloxy, amino, alkylamino, arylamino, aralkylamino, dialkylamino, alkanoylamino, thiol, alkylthio, ureido, nitro, cyano, carboxy, carboxyalkyl, carbamyl, alkoxycarbonyl, alkylthiono, arylthiono, arylsulfonylamine, sulfonic acid, alkysulfonyl, sulfonamido, aryloxy and the like. The substituent may be further substituted by hydroxy, halo, alkyl, alkoxy, alkenyl, alkynyl, aryl or aralkyl.

The term “heteroaryl” refers to an optionally substituted, aromatic group for example, which is a 4 to 7 membered monocyclic, 7 to 11 membered bicyclic, or 10 to 15 membered tricyclic ring system, which has at least one heteroatom and at least one carbon atom-containing ring.

Pharmaceutical compositions provided herein are formulated to be compatible with their intended route of administration. Exemplary routes of administration include, e.g., parenteral, intravenous, intramuscular, intradermal, subcutaneous, oral, inhalable (e.g., by mouth or nasal), transdermal (topical), and transmucosal.

Solutions or suspensions used for parenteral, intradermal, or subcutaneous application can include, a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. The parenteral preparation can be enclosed in ampoules, disposable syringes or multiple dose vials made of glass or plastic.

In further aspects, compositions provided herein can be formulated as tablets, capsules or elixirs for oral administration or as sterile solutions or suspensions for injectable administration.

In some aspects, a compounds described herein can be formulated as a sustained release composition which provides for slow, sustained release of the compound by a desired mode of administration. Such formulations can take the form of a sustained release gel, cream, suppository, capsule, or the like. In some aspects, active compounds are formulated within a system of carriers and excipients that protect the compound against rapid elimination from the body. Methods for preparation of such formulations are known to those skilled in the art.

Pharmaceutical compositions provided herein can also be utilized in conjunction with a delivery device. In some aspects, pharmaceutical compositions provided herein can be delivered via an intranasal spray, by inhalation, and/or by an aerosol. Methods for delivering pharmaceutical compositions directly to the lungs and/or nasal mucosa via nasal and/or pulmonary aerosols, as understood by those of ordinary skill in the pharmaceutical arts.

In some aspects, pharmaceutical compositions provided herein are delivered via a liposomal nanoparticle formulation. In an example embodiment, the compound can be formulated within the liposomes to enhance solubility and/or permeability across viral membranes. Liposomal formulations can be prepared according to methods known to those skilled in the art.

Pharmaceutical compositions suitable for injectable use include sterile aqueous solutions (for water soluble compounds) or dispersions and sterile powders for the extemporaneous preparation of sterile injectable solutions or dispersions.

Oral compositions generally include an inert diluent or an edible carrier, and can be incorporated with excipients in the form of tablets, troches, pills, capsules, or the like. Oral compositions can also be prepared using a fluid carrier for use as a mouthwash or rinse wherein the compound in the fluid carrier is applied orally and swished and expectorated or swallowed.

In some aspects, pharmaceutical compositions provided herein are formulated in dosage unit form (physically discrete units comprising a unitary, predetermined quantity of active compound calculated to produce the desired therapeutic effect in association with the required pharmaceutical carrier). The exact specifications of dosage unit forms will be dictated by the unique characteristics of the active compound, the particular therapeutic effect to be achieved, the preferred route of administration, and the like.

The term “therapeutically effective” means an amount of an antiviral compound provided herein (an effective dose) that can range from 0.0001 to 10000 mg/kg of body weight. Skilled artisans will appreciate that a variety of factors can influence the dosage required to effectively treat a subject, including but not limited to the severity of the disease or condition being treated, history of previous treatments, general health and/or age of the subject, and the like. Accordingly, exact dosages for any particular subject will typically be determined empirically.

Toxicity and therapeutic efficacy antiviral compounds provided herein can be determined using standard pharmaceutical procedures in cell cultures or experimental animals. For example, established methods can be used to calculate LD50 (the dose lethal to 50% of the population) and/or ED50 (the dose therapeutically effective in 50% of the population) doses for the antiviral compounds. The dose ratio between toxic and therapeutic effects is the therapeutic index, which can be expressed as the ratio LD50/ED50. Compounds which exhibit large therapeutic indices are generally preferred, as are formulations and modes of administration which enhance the therapeutic index for a particular compound.

Data obtained from the cell culture assays and animal studies can be used in formulating a range of dosage for use in humans. In some aspects, dosages of antiviral compounds provided herein lie within a range of circulating concentrations which include the ED50 with little or no toxicity. Dosages can vary within this range depending upon the dosage form, route of administration, and the like.

Therapeutically effective doses of compounds provided herein can be estimated initially from cell culture assays, e.g., based on the IC50 (the concentration of a test compound which achieves a half-maximal inhibition of viral activity and/or infection). For example, the IC50 observed in cell culture assays can be used to formulate a working dosage range for use in animal models in order to achieve a circulating plasma concentration range that includes the IC50 with little or no toxicity. Conversion factors and calculation methods for converting animal dosages to human dose estimates are well known in the pharmaceutical arts.

As used herein, “treating” includes prevention (prophylactic treatment), amelioration, alleviation, and/or elimination of a disease, disorder, or condition being treated or one or more symptoms of a disease, disorder, or condition being treated, as well as improvement in the overall wellbeing of a patient, as measured by objective and/or subjective criteria.

Compounds and methods provided herein are useful for treating and preventing (e.g., prophylactically treating) infections by any enveloped virus. “Enveloped” viruses are animal viruses having an outer membrane or ‘envelope’ comprised of a lipid bilayer with embedded viral proteins.

Also provided herein are methods of treating a disease or condition associated with an enveloped virus infection. For example, in some aspects, methods are provided herein for treating Crimean Congo hemorrhagic fever, Ebola, Marburg, Lassa fever, MERS, SARS, Nipah, and Rift Valley fever viruses and the like.

In addition, unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

### General Description

Viruses are categorized as either lipid-enveloped or non-enveloped. Enveloped viruses enter cells through membrane fusion of the viral and cellular membranes, then replicate within the host-cell, and then utilize specialized viral proteins to allow a process known as budding to occur. Once formed the bud will eventually be pinched off by membrane scission to release the enveloped particle to propagate the infection. Although the lipid membrane of enveloped viruses derives from the host cell, it differs from host cellular membranes in several biochemical and biophysical properties, such as biogenic reparative capacity, fluidity, lipid composition, and curvature. The technology disclosed herein is thus directed towards a method of treating such a broad-spectrum of enveloped viral pathogens because, as generally stated above, there is a need, in fact long-felt need, to provide for an effective vaccine or therapeutic approach to treat a broad spectrum of infectious viral agents effectively.

Accordingly, because of such a long-felt need, the embodiments herein in an exemplary manner, capitalize on such aforementioned biochemical and biophysical properties. As an example, it is to be specifically noted that the membranes of budding viral particles are highly curved relative to the membranes of much larger host cells. As a result, the fusion of enveloped viral particles with new host cells requires that the high curvature viral membranes undergo elastic stresses and subsequent negative curvature needed to promote fusion between the outer lipid monolayers of the viral particles and host cell membranes.

This central role of virus-host cell membrane fusion during viral entry into host cells information is important with respect to the antiviral therapeutic methodologies disclosed herein. There are several factors which can play a role such as intercalation, modification, or otherwise modifying the binding of viral membranes with the goal of disturbing the membrane dynamics required for successful virus-host cell fusion. Fortunately, while cells have well-developed membrane repair mechanisms, viruses do not. Such mechanisms enable compounds, such as compounds disclosed herein, to be utilized in a manner that affect viral membranes while not significantly affecting cell membranes. A resultant benefit of targeting viral membranes instead of viral proteins is the relatively lower chances for viruses to acquire genetic mutations that will render them resistant to the antiviral. Typical antivirals target viral proteins, and viruses can relatively more easily develop resistance to the antivirals by acquiring genetic mutations.

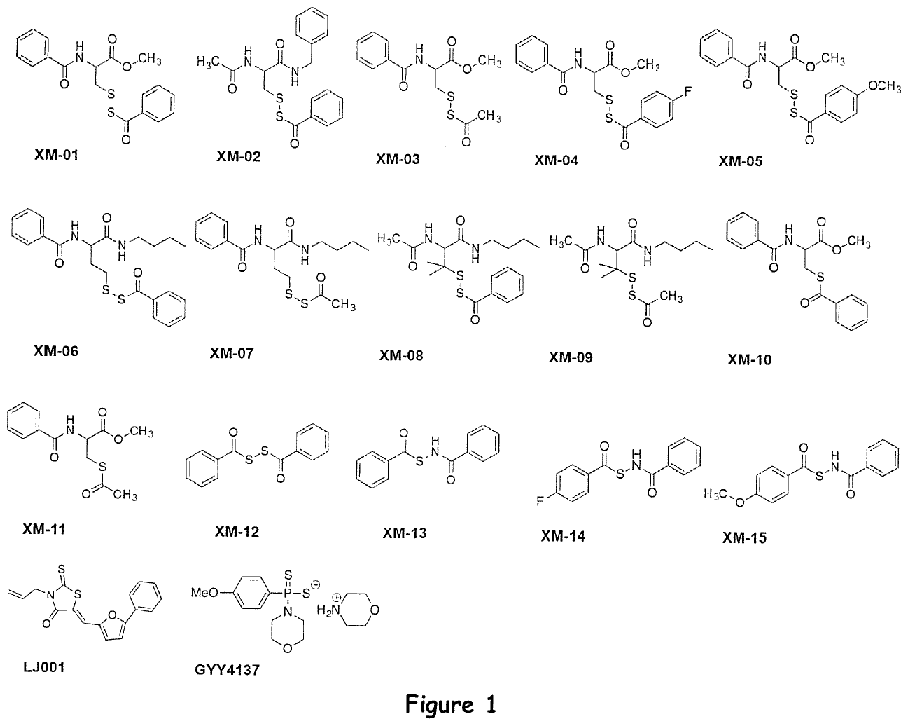

As part of the development for the therapeutic and/or preventive uses of the compounds disclosed herein, a library of H₂S and/or sulfur-radical donor compounds were tested for their use as broad spectrum antivirals. Included in the library were a noted number of beneficial antiviral compounds that contained sulfur-sulfur (S—S) bonds as seen in FIG. 1. The mechanism of action of the tested compounds are similar to LJ001 in that its principle mechanism of action is against viral membranes and it produces radicals at the membrane. But unlike LJ001 that requires light exposure, the disclosed compounds utilize sulfur radical-releasing mechanisms to disrupt the viral membrane and as a result can be utilized without activation from a light source.

In a surprising and unexpected outcome of the methodologies herein, the disclosed invention demonstrates the ability to inhibit viral infections by several important enveloped viruses even in the absence of light. The disclosed invention thus represents methodologies using referenced antiviral compounds that targets an essential part of the virus replication, i.e, membrane fusion. Seeing as this function is absolutely essential for viral entry into host cells for enveloped viruses, the disclosed compounds, in the use manner disclosed herein, is superior over currently available alternatives on the market. Additionally, the highly desirable characteristics that the disclosed compounds exhibit make them a new class of sought-after multi-functional antiviral compounds.

### Specific Description

Compounds of note in the present invention are thus sulfur-based compounds. Information on similar compounds as utilized herein can be found in U.S. Pat. No. 9,096,504, entitled: “Controlled Chemical Release of Hydrogen Sulfide,” to Xian Et al. issued Aug. 4, 2015, the contents of which is incorporated herein by reference in its entirety. All stock solutions for these compounds were prepared in 100% DMSO, stored at −20° C., and used within 6 months of reconstitution.

Cell Culture.

293T and PK13 cells (ATCC) were cultured in Dulbecco's modified Eagle's medium with 10% fetal bovine serum (FBS). Vero cells (ATCC) were cultured in minimal essential medium alpha with 10% FBS. Human lung epithelial cells (A549, ATCC) were grown in complete DMEM containing 10% FBS, 100 IU/ml penicillin, 100 μg/ml streptomycin (Gibco). MA104 cells were obtained from ATCC and grown in Dulbecco's modified Eagle's medium with 10% FBS, 100 IU/ml penicillin, 100 μg/ml streptomycin (Gibco).

### Cells and Cell Viability Assay

Vero cells (ATCC) were used to measure cytotoxicity using the Cell Counting Kit (CCK-8) according to the manufacturer's instructions. Reaction plates were incubated for 90 min and absorbance was read at 450 nm using a Tecan microplate reader (Infinite M-1000). The quantity of formazan dye produced, when WST-8 is reduced by dehydrogenases, is directly proportional to the number of living cells, allowing the measurement of cell viability.

### Cell-Cell Fusion Quantification

293T cells grown in 6-well plates were transfected at 70-90% confluency with NiV F and G DNA expression plasmids (1:1 ratio, 2 μg total DNA per well) using lipofectamine 2000. 18 hours post-transfection cells were fixed in 0.5% paraformaldehyde (PFA) and syncytia was counted under an inverted microscope (200×) with a syncytium defined as four or more nuclei within a single cell. Five fields per well were counted for each experiment.

Receptor ephrinB2 and Antibody Binding were Measured by Flow Cytometry

For receptor binding, 293T cells were transfected with 2 mg NiV G expression plasmids and collected 24-hours post-transfection. Collected cells were then incubated with compound for 30 min followed by incubation with soluble ephrinB2 at 100 nM for 1 hour. This was followed by two washes with FACS buffer (1% FBS with PBS) and incubation with a fluorescent anti-human Alexa Fluor 647 antibodies diluted 1:200 for 30 min at 4° C., follow by two washes. Cells were fixed in 0.5% PFA and read on a flow cytometer.

### Transmission Electron Microscopy (TEM) Imaging

5 μL of VLP or VSV—NiV suspension were pipetted onto a 200-mesh Formvar-coated nickel grid and allowed to settle for 20 min at room temperature. Excess liquid was removed by wicking with filter paper before coating the deposited sample with 5 μL of 1% Uranyl Acetate (UA). After 2.5 minutes, excess UA was wicked off using filter paper and dried overnight in a desiccator. TEM micrographs of the samples were recorded under high vacuum with electron beam strength set at 200 kV using the FEI Technai G2 20 Twin TEM.

### Testing Compound XM-01 Effects on Triggering of NiV—F

F-triggering assays were performed with some optimizations over what is commonly known in the art. PK13 cells were transfected with NiV—F expressed in a pCAGGS plasmid, NiV-G expressed in a pcDNA3.1 plasmid, and green fluorescent protein (GFP) expressed in a pMAX plasmid at a 13:6:1 ratio, respectively. At 24 h post-transfection, cells were mixed with untransfected PK13 (negative control) or PK13B2 (receptor expressing) cells at a 1:1 ratio and incubated for 60 minutes at 4° C. or 37° C. in the presence of HR2-Cy5 peptide and 1M of DMSO (vehicle control) or compound XM-01. Subsequently, cells were brought out of solution and re-suspended in FACS buffer (1% heat inactivated FBS in phosphate buffered saline [PBS]). HR2-Cy5 peptide bound to trigger NiV—F was detected with the Cy5 fluorophore by flow cytometric analysis.

### Cytotoxicity Assay

Vero cells were incubated with each compound for 30 min to 24 hours, as indicated, at the indicated concentrations. This was followed by an incubation with a cell counting kit reagent (CKK-8) for 1-2 hours, and then the measurement of absorbance at 450 nm in an infinite M100 microplate reader. The quantity of the formazan dye produced when WST-8 is reduced by dehydrogenases is directly proportional to the number of living cells.

### Membrane Fluidity Assay

Virus particles were incubated with the indicated compound for 30 min, followed by a 1 hr fluorescent lipid reagent incubation. The reagent was obtained from membrane fluidity kit: ab 189819. Virus particles were washed with NTE buffer (1% FBS with PBS), and resuspended in NTE buffer (NaCl, Tris-HCl, EDTA). Subsequently, the fluorescence intensity for the monomer and excimer was read on an infinite M100 microplate reader.

### Pseudotyped Virus Production

Pseudotyped virions containing NiV F and NiV G were manufactured as previously described. 18 Briefly, 15-cm plates of 293T cells were transfected at 37° C. with NiV F and NiV G expression plasmids at a 1:1 ratio. 8 hr post-transfection media was switched to fresh growth media. After an additional 16-hr the cells were infected with recombinant VSV-G-rLuc. 2-hr later the infection media was removed and replaced with growth media. 24-hours after infection virions were harvested from cell supernatants using ultracentrifugation, re-suspended in NTE buffer with 5% sucrose, and stored at −80° C. in 100 μl aliquots.

### Detection of Protein Conformations by Flow Virometry

Pseudotyped NiV (pNiV) virions were incubated for 30 min with compound XM-01 at 40° C., then washed by ultracentrifugation with NTE buffer (150 mM NaCl, 40 mM Tris-HCl at pH 7.5, and 1 mM EDTA) at 35,000 rpm for 2 h. Treated virus was resuspended in NTE buffer, then stained basically as previously described. Anti-NiV—F and/or anti-NiV-G specific rabbit 1o antibodies (Anti NiV—F Ab 66, or anti NiV-G Ab 213)20-22 at 1:100 dilution were used for 1 hr followed by a FACS buffer (1% FBS in PBS) wash and incubation with 2 antibody Alexa 647 goat and anti-rabbit for 30 min followed by one more FACS buffer wash. Relative levels of antibody binding were measured by a flow virometry technique, using a Guava easyCyte8HT flow cytometer. Background mean fluorescence intensity (MFI) obtained by binding equal concentrations of primary and secondary reagents to mock virus obtained by transfecting 293T cells with the PCDNA3.1 backbone DNA expression vector were then subtracted from the MFI of pseudotyped NiV/VSV virions.

### Pseudotyped NiV/VSV Viral Infection Assays

Virus particles were incubated for 30 min with or without the indicated amounts of compound or the corresponding vehicle DMSO control. Then, Vero cells were infected with 10-fold dilutions of pseudotyped virus particles in infection buffer (PBS+1% FBS) and incubated for 2 hr at 37° C. After 2 hr growth media was added. 18-24 hr post-infection cells were lysed and an infinite M100 microplate reader was used to measure luciferase activity.

Plaque assay for measurement of HSV-1 infections. At 18 to 24 hr post-infection, culture medium was removed, and cells were fixed with ice-cold methanol-acetone solution (2:1 ratio) for 20 min at 20° C. and air dried. Virus titers were determined by immunoperoxidase staining with anti-HSV polyclonal antibody HR50.

### Plaque Assay for Measurement of RSV Infections

Human respiratory syncytial virus (RSV A2 strain) was propagated on CV 1 cells (ATCC) and purified by centrifuging two times on discontinuous sucrose gradients. XM-01 compound (10 or 30 μM as indicated) was incubated with purified RSV at room temperature for 45 min or 2 hr before infecting A549 cells at multiplicity of infection (MOI) of 0.5 or 0.01. Briefly, the XM-01 compound pre-treated RSV was adsorbed onto the cells in serum-free, antibiotic-free OPTI-MEM medium (Gibco) for 1.5 h at 37° C. Following adsorption, A549 cells were washed with PBS and the infection was continued for 16 hr in the presence of XM-01 compound before collecting the supernatant. A plaque assay was performed to determine the viral titer (pfu/ml) in the collected supernatant. Briefly, CV-1 cells were infected with serial dilutions of the culture supernatant in a 12-well plate as described above. After 1.5 h the cells were washed with PBS and medium was replaced with 1% methylcellulose in complete growth medium. Plaques were stained after 24-48 hr with 1% crystal violate and counted to determine the viral titer.

### Rotavirus Infection Assays.

18β-glycyrrhetinic acid (GRA) stock solutions were prepared to a concentration of 100 mg/mL in DMSO and aliquots were stored at −80° C. Stock solutions were diluted to working concentrations in DMEM without FBS. For the control compounds, MA104 (ATCC) cells were treated for six hours with 25 μg/ml GRA. Viability was measured with the Promega CellTiterGlo Assay according to the manufacturer's protocol, with digitonin as the control for 100% cytotoxicity. Data shown are representative of two experiments, with each concentration tested in triplicate in each experiment. Error bars indicate SEM. To test compound XM-01, after MA104 cells were infected with 8.9×105 ffu/well of trypsin-activated bovine rotavirus strain NCDV. Mock-infected wells received 50 μl of 0% M199 vehicle media. 50 μl of fresh 2× control and experimental compounds were added and at 18 hours post-infection, the cells were fixed for 10 minutes with 80% acetone.

### XM-01-Like Compounds Inhibit Pseudotyped NiV Infections

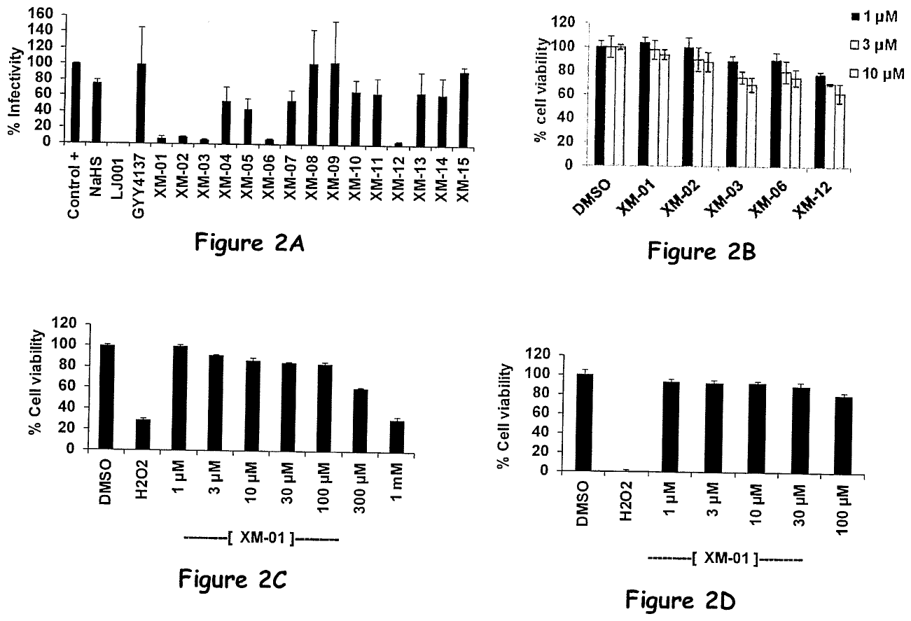

A library of perthiol donors and thiol donors where screened, while comparing them with the known hydrogen sulfide donors NaHS, GYY-413728, and LJ001, a known control inhibitor of viral entry. Compounds where screened as antiviral inhibitors using the well-established high-throughput pseudotyped NiV/VSV virus (pNiV) infection system. Briefly, pseudotyped virions were incubated with the respective compound for 30 min, and then the mix was used to infect Vero cells for 2 hr, after which fresh media was added. Luciferase activity was detected 18-24 hr post-infection using a Tecan fluorimeter plate reader. Interestingly, the five compounds that best inhibited (pNiV) infections contained pseudo-perthiol bonds as seen in FIG. 1 and FIG. 2A. The disclosed compounds where then examined in a cytotoxicity assay using a CKK8 cytotoxicity kit that measures dehydrogenase enzymatic activity as seen in FIG. 2B. XM-01 was selected for further studies as it showed low cytotoxicity at 1, 3, and 10 μM concentrations, and high inhibition of infectivity at 10 μM. The cytotoxic effects of XM-01 at concentrations between 1-1 mM are shown, and the CC50 calculated using Graphpad Prism was close to 1 mM as seen in FIGS. 2C and D.

### Compound XM-01 Inhibits Four Distinct Enveloped Viruses but not a Non-Enveloped Virus

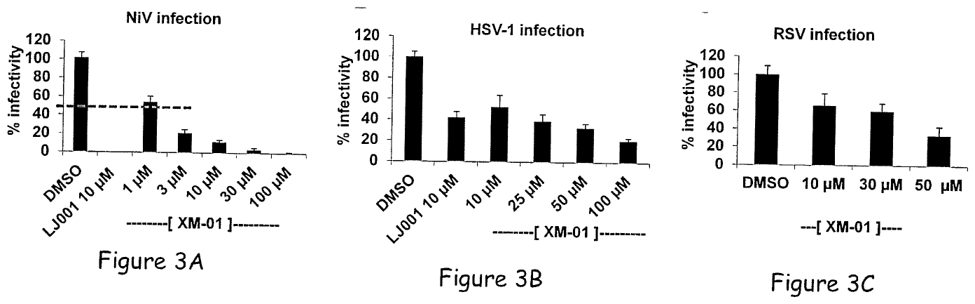

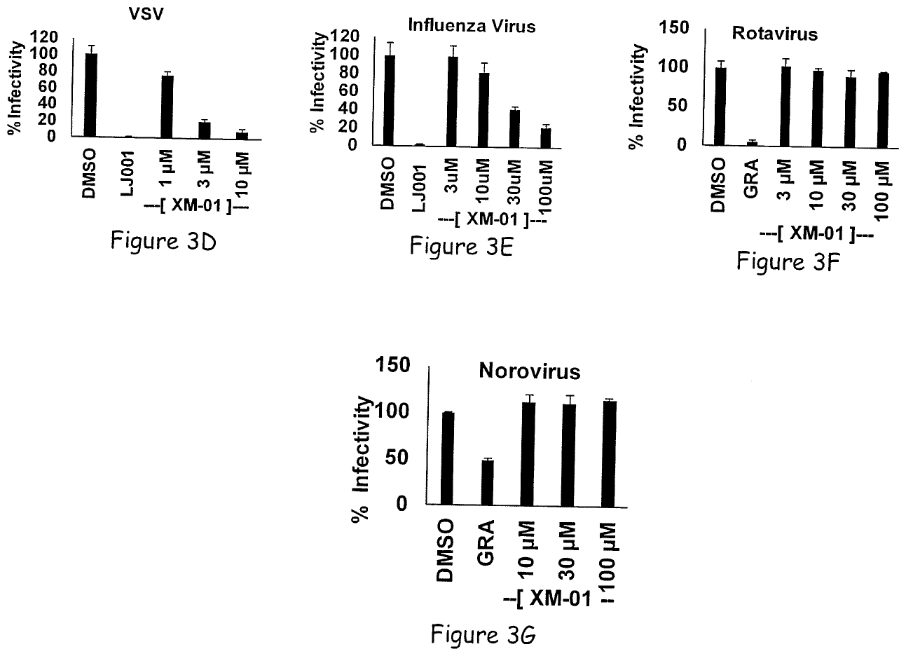

Pseudotyped NiV (pNiV), and live HSV-1, RSV, VSV or Influenza viruses were inhibited with XM-01, as seen in FIGS. 3B-3E. Using the pNiV system, XM-01 showed a low micromolar inhibitory concentration (EC50˜1 μM), and approximately a 3 log difference between this inhibitory and the cytotoxic concentrations (CC50-1 mM), as seen in FIG. 2C and FIG. 3A. The results disclose that compound XM-01 inhibited pNiV infections well at 10 μM, various concentrations where examined for other enveloped virus infections. By way of example, XM-01 is disclosed to inhibit HSV-1 plaque formation in a concentration-dependent fashion, as seen in FIG. 3B. Briefly, XM-01 was incubated for 30 min with HSV-1 KOS (100 PFU/well) and then the mix was added to Vero cells. At 3 hr post inoculation the medium was removed, extracellular virus was acid-inactivated, and plates were incubated for 24 hr. Plaques were detected by immunoperoxidase staining and quantified. Similar experiments indicated that respiratory syncytial virus (RSV), vesicular stomatitis virus (VSV), and Influenza virus infections were also inhibited by XM-01 as seen in FIG. 3C-3E. In contrast, there was no significant inhibition of a non-enveloped rotavirus infection as seen in FIG. 3F or norovirus as seen in FIG. 3G. All data shown are by means of triplicate experiments with the standard errors. Collectively, the disclosed results indicate that XM-01 inhibits and effectively treats enveloped viruses selectively while not interfering with non-enveloped viruses.

### XM-01 Inhibits Virions Directly, Early in the Infection Process, and in the Absence of Light

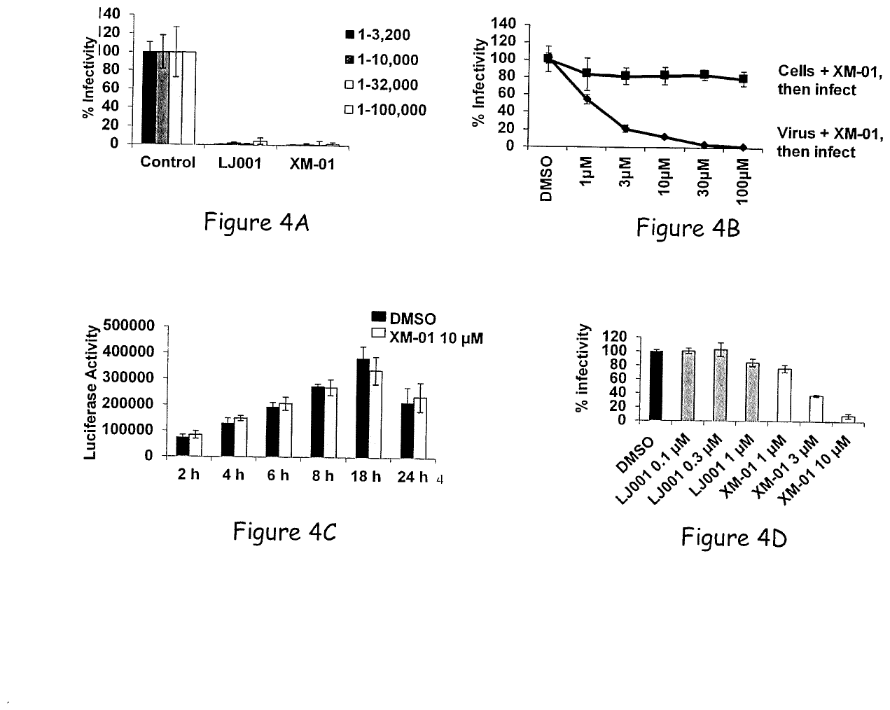

To elucidate the mechanism of action for XM-01 and as a result similar compounds disclosed herein, cells were infected with different virus dilutions, however, pNiV virions were first treated with XM-01 for 30 minutes and then washed by ultracentrifugation with phosphate buffer saline (PBS) to exclude unbound XM-01. This was followed by infection of Vero cells. The resulting virions were highly inhibited in their capability to infect Vero cells as compared to the DMSO virus control, indicating that compound XM-01 acts directly on the virus, and not on the cells as seen in FIG. 4A. As part of the process for corroboration, cells were pretreated with XM-01 for 30 min and washed away XM-01 using PBS, before infecting the cells with untreated virions. In contrast to the effects of XM-01 on the virions in FIG. 4A, and as indicated at the bottom of the curve shown in FIG. 4B, the compound did not significantly reduce viral infection as seen in FIG. 4B at the top of the curve. Such experiments confirmed that XM-01 acts directly on the virions in a prophylactic/inhibitory nature.

To begin elucidating the step(s) of infection affected by XM-01, a time of XM-01 addition experiment was performed, as seen in FIG. 4C. Vero cells where infected with virions for 2, 4, 6, 8, 18, or 24 hr. After infection, the unbound virus were washed away from the cells three times with PBS, followed by addition of XM-01 in cell growth media. The cells where then incubated for the additional time necessary to complete a 24 hr period after initial virus exposure and then detected the infection levels by measuring luciferase activity. It is to be first noted that the longer the virus was allowed to infect the cells, the higher the level of infection observed, consistent with longer periods-of-time allowing more virus to contact, bind, and enter cells. Second, and most importantly, after any given length of time of infection, XM-01 was completely incapable of inhibiting the virus as compared to infections with the respective DMSO vehicle control. These results surprisingly and unexpectedly show that once a virus has entered a cell, XM-01 is incapable of exerting its inhibitory/preventative/prophylactic activity, consistent with XM-01 inhibiting a viral entry step as seen in FIG. 4C.

It is also to be appreciated that XM-01 did not need light to inhibit pNiV infections. By contrast, the inhibitory activity of our control LJ001 as a broad-spectrum antiviral was severely reduced in the absence of light, as seen in FIG. 4D. The activity of XM-01 in the absence of light was an unexpected but an important result for the potential future uses of XM-01 as a broad spectrum antiviral therapeutic.

### XM-01 Inhibits Membrane Fusion

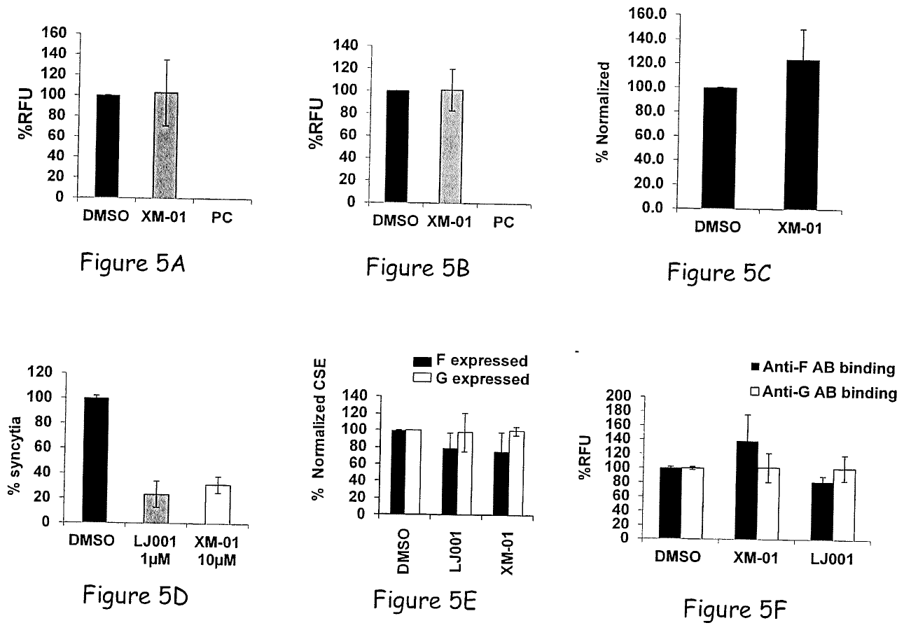

As the viral components that most affect viral entry are the viral glycoproteins and the viral membrane, several experiments where conducted to investigate whether XM-01 affects the viral glycoproteins. First, to analyze whether XM-01 affected viral binding to cell receptors, 293T or PK13 cells were transfected with a NiV-G expression plasmid, followed by an incubation with soluble receptor ephrinB2 and XM-01. After a 1 hr incubation at 40° C., binding of ephrinB2 receptor to NiV G was measured by flow cytometry. XM-01 did not interfere with the receptor binding ability of the NiV G glycoprotein, as seen in FIG. 5A and FIG. 5B. Furthermore, XM-01 did not affect the NiV fusion (F) protein's ability to be triggering by G to execute membrane fusion, as shown by our F-triggering assay, as seen in FIG. 5C and Figures E-F. Briefly, PK13 cells where transfected with NiV G and F expression plasmids to observe if XM-01 would trigger the NiV—F protein to decrease. The cells were incubated with the transfected PK13 cells, which express the ephrinB2 receptor, for 1 hr at 4° C. to synchronize NiV G-receptor binding. XM-01 was then added to the transfected cell and a HR2 peptide known to bind NiV F only after it has been triggered by NiV G to execute membrane fusion. The HR2 peptide was conjugated to a Cy5 flourophore for flow cytometric detection (HR2-Cy5). After addition of XM-01 and HR2-Cy5, the cells where incubated at a temperature of 37° C. to allow F-triggering to occur. We observed that the NiV—F protein triggering was not decreased upon XM-01 treatment, as seen in FIG. 5C.

With the observation that XM-01 did not affect the function of G or F protein functions. The function of cell-cell fusion was the next promising cellular function to examine. Cell-cell fusion is both a surrogate assay used to study viral-cell membrane fusion executed by paramyxoviral glycoproteins as well as a significant outcome of paramyxoviral infections, and is also considered important for viral spread between infected and naïve cells. As such therapeutics that target this function are executed by glycoproteins and it was shown that XM-01 affected cell-cell membrane fusion, as seen in FIG. 5D. 293T cells where transfected with NiV F and G expression plasmids were allowed to incubate with XM-01 for 18 hr post-transfection. Nuclei within syncytia were counted and compared to the DMSO control. These results are consistent with XM-01 interfering with a step on the membrane fusion cascade, and demonstrate that the XM-01 affects viral entry in the host cell. The inhibition of the cell-cell fusion by XM-01 was further demonstrated by decreasing the surface expression of F and G glycoproteins, with the surface expression being measured by flow cytometry. The end result of these results showed no significant affect demonstrating that XM-01 does not affect binding or F-triggering steps of membrane fusion, as seen in FIG. 5 D.

XM-01 also did not significantly affect the glycoprotein conformations as seen by FIG. 5E. In this experiment, pseudotyped NiV was incubated with XM-01 for 30 min before the compound was washed away with NTE buffer. Primary and secondary antibodies were allowed to bind. We then measured the relative binding levels of two polyclonal antisera to NiV F or NiV G by flow cytometry and observed no significant changes in antibody binding to either glycoprotein. As a control, we used the LJ001 compound known to affect viral membranes but not viral glycoprotein conformations, as shown in FIG. 5E. Surprisingly and unexpectedly, all of the above results suggest that XM-01 affects the important process of membrane fusion, without significantly affecting glycoprotein functions as seen in FIG. 5F.

### Compound XM-01 Compromises Viral Membranes

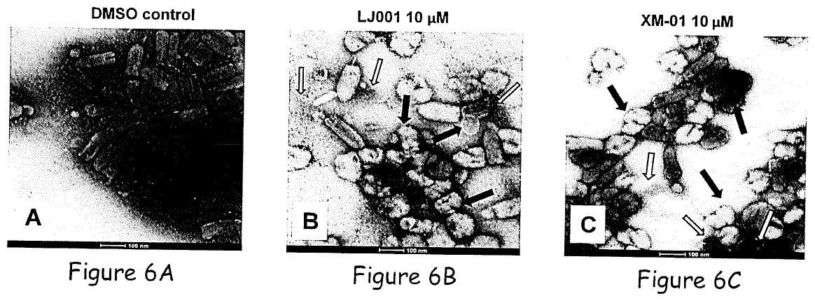

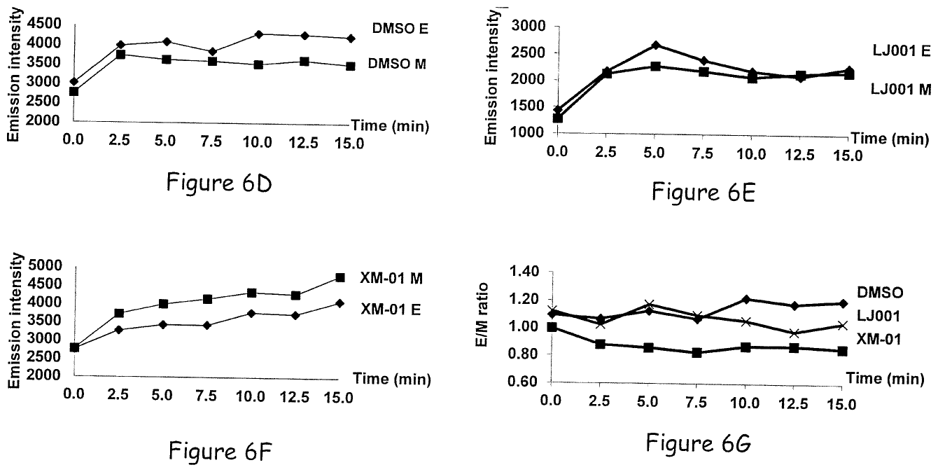

XM-01 clearly acts on steps after binding and F-triggering, and appeared to affect the virus particles directly, but not the glycoproteins. Furthermore, XM-01 affected several enveloped viruses tested, but not a non-enveloped virus. Such results indicated that XM-01 affects the viral membranes and as a result it can be used as a new and novel class of broad spectrum antivirals. Thereafter, images were utilized to explore how XM-01 may physically affect the virions. It was evident that viruses treated with XM-01 had a compromised viral membrane as compared with the DMSO control, as shown by FIGS. 6A-C. LJ001-treated pNiV was again utilized to confirm the detection of compromised membranes, as LJ001 affects the viral membrane. It was also noted that RNA spilled more frequently from virions treated with XM-01 and LJ001 as compared to the DMSO control.

### Pyrene Molecules as Useful Lipid-Linked Fluorophores

In cell membrane studies, pyrene lipids are a tool to study lipid trafficking and metabolism, and membrane fluidity. The ratio of excimer formation to monomer labeled lipid provides information on cell membrane fluidity. Unexpectedly and surprisingly, the present methodology enables the detection of membrane fluidity on virus particles. A healthy bilayer phospholipid membrane showed a relative increase of the ratio of excimer to monomer for our DMSO vehicle control, which correlates with a detection of membrane fluidity. It is to be noted that the positive control LJ001 showed a reduction in the ratio of excimer to monomer, indicating an effect on membrane fluidity, as observed using other methods that measure membrane fluidity. The disclosed results showed that pNiV virions treated with XM-01 showed a considerably lower ratio of excimers to monomers than did the DMSO control, indicating that the viral membrane fluidity was affected by XM-01 as shown by FIG. 6D-G.

XM-01 has great inhibitory properties against envelope viruses such as NiV, VSV, RSV, HSV-1, just to name a few examples while not inhibiting rotavirus, a non-enveloped virus. Surprisingly, XM-01 showed relatively low cytotoxicity levels as compared to other perthiol-based and sulfur-sulfur bond compounds tested, with a CC50/EC50 ratio for XM-01 of about 1,000-fold. This coupled with the above disclosed embodiment shows that XM-01 is a safe, effective and novel broad-spectrum antiviral agent. Other example embodiments showed that XM-01 did not interfere with the viral glycoproteins or their functions tested, but inhibited viral entry via affecting the viral membranes and their fluidity. Notably, XM-01 does not need light in order to inhibit enveloped virus infections allowing for more flexibility in its use as a therapeutic agent. By way of comparison, the control broad-spectrum antiviral LJ1001 used requires exposure to light to be effective and has a very limited use as a therapeutic agent. Since XM-01 targets a key step in the entry process of all enveloped viruses into their host cells, membrane fusion, these characteristics make XM-01 and its derivatives great candidates as a broad-spectrum antiviral agents against enveloped viruses.

Compounds XM-01, 2, 3, 10, and 11 all showed high inhibitory properties and most of them showed manageable cytotoxic. Therefore, these compounds together with XM-01 constitute a new class of sulfur based broad-spectrum antivirals that inhibit viral membranes without the need of light for activation. The comparison of the structures of the active vs inactive compounds tested revealed that all XM-01-type compounds have the acyl disulfide core structure.

The disclosed embodiments indicate that XM-01 and similar analogs are much more potent as an antiviral inhibitor than other compounds known to those skilled in the art such as NaHS or GYY4137. As shown above, XM-01 inhibited enveloped viruses, but its activity is likely not linked to its H₂S release. To illustrate this point, the structure of XM-01 shown in FIG. 1 and activity of XM-01 was compared to NaHS or GYY4137, as shown in FIG. 2A. This is noteworthy do to that NaHS or GYY4137 are compounds known to have relatively higher levels of H₂S releasing activity, which is attributed to their inhibitory properties. The EC50 of GYY4137 has been shown to be 10 mM, an order of magnitude higher than the EC50 of XM-01, as shown by FIGS. 3A-G.

Additionally, XM-01 application as a broad spectrum antiviral is further exemplified by it having an effect on virus and not having an effect on viral glycoproteins. This indicates that this compound will likely not affect important cellular proteins, which explains its low observed cytotoxicity levels. Also as stated, viruses do not have the capacity to repair there membranes but by way of comparison, cells have a high capacity for repairing their membranes. This also means that viruses are less likely to mutate their genes (all encoding for viral proteins) to become resistant to this type of compound, as compared to antiviral inhibitors that target viral proteins. It is also unlikely that a virus would be able to mutagenize its genes to modify the viral membrane, which is cell derived, which would be another way to create viral resistance to a membrane inhibitor.

Finally, XM-01 does not require light in order to inhibit an enveloped virus, which makes it far more practical and applicable as a therapeutic antiviral. When compared, with other known antiviral in the art such as LJ001, which does require light to inhibit a viral membrane. LJ001 is being explored as an inhibitor of viral transmission through water, as water is transparent. However, for places within the body where light does not easily penetrate, XM-01-like compounds are a better option.

Many enveloped viruses highly affect human and animal health and some have a high potential to cause global pandemics. As such, it is urgent to develop promising new types of broad-spectrum membrane fusion inhibitors. XM-01 represents one such effort and provides a new type of novel broad spectrum antiviral compounds. Since XM-01 does not appear to affect viral glycoproteins functions or conformations, XM-01 type compounds hold promise as vaccine adjuvants, as conformationally intact viral glycoproteins are typically excellent at eliciting immune responses.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof with the spirit and scope of the description provided herein.

1-15. (canceled)

16. A method of inhibiting cell entry of one or more enveloped type viruses into a host cell, comprising the step of contacting the enveloped type virus with a compound having the chemical formula: 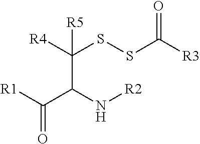
or a pharmaceutically acceptable salt thereof, wherein:
R1 is selected from the group consisting of OH, OR6, NHR6, and NR6R7 where
R6 and R7 are selected from the group consisting of alkyl, aryl, methyl and heteroaryl;
R2 is selected from the groups consisting of OR6, acyl, alkyl, aryl, and sulfonyl;
R3 is selected from the group consisting of OR6 alkyl, aryl, substituted aryl, and heteroaryl; and
R4 and R5 are independently selected from the groups consisting of hydrogen, methyl or alkyl, substituted alkyl, aryl, and substituted aryl,
wherein the compound disrupts a viral membrane of said one or more enveloped type viruses and prevents or inhibits virus-host cell membrane fusion.

17. The method of claim 16 wherein contacting occurs in vivo in a mammal, wherein said compound is provided to the mammal at a therapeutic dose which enables said step of contacting to occur within said mammal.

18. The method of claim 16 wherein contacting occurs in vivo in a human, wherein said compound is provided to the mammal at a therapeutic dose which enables said step of contacting to occur within said human.

19. The method of claim 16 wherein the one or more enveloped type viruses is selected from the group consisting of Nipah Virus (NiV), vescular stomatitis virus (VSV), respiratory syncytial (RSV), herpes simplex virus type I (HSV-1), rotavirus, and influenza virus.

20. The method of claim 16 wherein the one or more enveloped type viruses is selected from the group consisting of hepatitis C virus, yellow fever virus, respiratory syncytial virus, sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, ebola virus, marburg virus, La Crosse virus, California encephalitis virus, Hantaan virus, Crimean-Congo virus, Rift Valley fever virus, Lassa fever virus, Argentine hemorrhagic fever virus, Bolivian hemorrhagic fever virus, Colorado tick fever virus, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

21. The method of claim 16, wherein R1 is OR6, wherein R6 is methyl.

22. The method of claim 16, wherein R2 is OR6 wherein R6 is aryl.

23. The method of claim 16, wherein the disrupting of the viral membrane occurs without activation from a light source.

24. A method of providing a subject with a broad spectrum treatment of protection from at least two different enveloped type viruses, comprising the step of administering to a subject that is infected with said at least two different enveloped type viruses or who will be exposed to said at least two different enveloped type viruses a compound having the chemical formula: 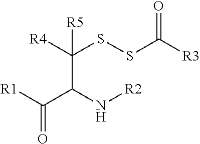
or a pharmaceutically acceptable salt thereof, wherein:
R1 is selected from the group consisting of OH, OR6, NHR6, and NR6R7 where
R6 and R7 are selected from the group consisting of alkyl, aryl, methyl and heteroaryl;
R2 is selected from the groups consisting of OR6, acyl, alkyl, aryl, and sulfonyl;
R3 is selected from the group consisting of OR6 alkyl, aryl, substituted aryl, and heteroaryl; and
R4 and R5 are independently selected from the groups consisting of hydrogen, methyl or alkyl, substituted alkyl, aryl, and substituted aryl.

25. The method of claim 24 wherein said at least two different enveloped type viruses are each selected from the group consisting of rotavirus, hepatitis C virus, yellow fever virus, respiratory syncytial virus, sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, ebola virus, marburg virus, La Crosse virus, California encephalitis virus, Hantaan virus, Crimean-Congo virus, Rift Valley fever virus, Lassa fever virus, Argentine hemorrhagic fever virus, Bolivian hemorrhagic fever virus, Colorado tick fever virus, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

26. The method of claim 24, further comprising providing said subject with a second therapeutic to the subject, said therapeutically effective dose of said compound and said second therapeutic being provided separately or as a combined preparation for simultaneous, separate, or sequential use.

27. The method of claim 26, wherein the second therapeutic agent being in a form of a pharmaceutically acceptable salt.

28. A method of treating or inhibiting a viral infection caused by an enveloped type virus selected from the group consisting of rotavirus, hepatitis C virus, yellow fever virus, respiratory syncytial virus, sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, ebola virus, marburg virus, La Crosse virus, California encephalitis virus, Hantaan virus, Crimean-Congo virus, Rift Valley fever virus, Lassa fever virus, Argentine hemorrhagic fever virus, Bolivian hemorrhagic fever virus, Colorado tick fever, JC virus, BK virus, human cytomegalovirus, varicella-zoster virus, human adenovirus, HTLV-1, HTLV-2, and human parvovirus, comprising administering to a subject a therapeutically effective dose of compound having the chemical formula: 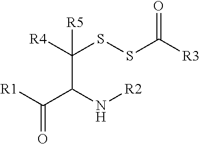
or a pharmaceutically acceptable salt thereof, wherein:
R1 is selected from the group consisting of OH, OR6, NHR6, and NR6R7 where
R6 and R7 are selected from the group consisting of alkyl, aryl, methyl and heteroaryl;
R2 is selected from the groups consisting of OR6, acyl, alkyl, aryl, and sulfonyl;
R3 is selected from the group consisting of OR6 alkyl, aryl, substituted aryl, and heteroaryl; and
R4 and R5 are independently selected from the groups consisting of hydrogen, methyl or alkyl, substituted alkyl, aryl, and substituted aryl.

29. The method of claim 28, further comprising administering a second therapeutic to the subject, said therapeutically effective dose of said compound and said second therapeutic being provided separately or as a combined preparation for simultaneous, separate, or sequential use.

30. The method of claim 29, wherein the second therapeutic agent being in a form of a pharmaceutically acceptable salt.
